(19) United States
(12) Patent Application Publication
(10) Pub. No.: US 2007/0112423 A1
(43) Pub. Date: May 17, 2007

(54) Devices and methods for treatment of venous valve insufficiency

(76) Inventor: Jack F. Chu, Santa Rosa, CA (US)

Correspondence Address: DEANNA J. SHIRLEY, 3418 BALDWIN WAY, SANTA ROSA, CA, 95403, US

(21) Appl. No.: 11/281,769

(22) Filed: Nov. 16, 2005

(51) Int. Cl. A61F 2/24

(52) U.S. Cl. 623/2.11; 623/2.36

(57) ABSTRACT

Devices and methods for improvement of functioning of a valve of a subject are disclosed. A device exhibiting an outward bias at its proximal end and an outward bias at its distal end, disposed at an angle to the bias at the proximal end is described. A device having a first arm and a second arm separated by a peak and having shoulders is also described. Alternative embodiments which function to decrease the distance between valve leaflets, having a first arm and a second arm biased toward one another are also disclosed. Several embodiments which may have mirror image ends are also disclosed. Any of the devices may have barbs, umbrella structures, sutures, or a variety of spring elements. Devices may be implanted surgically, percutaneously or subcutaneously. Methods and devices for delivery and deployment of devices are disclosed as well as methods for treatment of a valve of a subject.

### FIELD OF THE INVENTION

The invention herein is related to implantable medical devices and more specifically to devices and methods for treatment of venous valve defects, including resulting chronic venous insufficiency.

### BACKGROUND OF THE INVENTION

The healthy valves of a vein open and close to facilitate the flow of blood through the body in substantially one direction back to the heart. Venous insufficiency is a common condition in which the valves of the veins are damaged, and/or the venous vessels of the legs are over-dilated, thereby preventing the proper closure of the valves to effect directional blood flow. As a result, the veins do not efficiently return blood from the lower limbs of the body to the heart. Chronic venous insufficiency is a condition in which prolonged insufficient venous circulation results in pooling of blood in the legs and feet, leading to swelling, changes in skin color, and eventually ulcerations and deep vein thrombosis. Deep vein thrombosis involves the formation of a clot which may interfere with circulation, and may break off and travel through the blood stream, potentially lodging in the brain, lungs, heart, or other area, causing severe damage to the affected organ. Chronic venous insufficiency is a common disorder affecting between 2-5% of, or roughly 25 million Americans. It is estimated that 2 million workdays are lost annually in the United States and $1.4 billion is spent each year on this medical condition

The most common cause of chronic venous insufficiency is valve reflux, either primary or secondary. Primary reflux is a condition in which the valve leaflets are stretched, redundant and have a tendency to invert, allowing blood to flow in a reverse direction. In addition, the vein dilates, widening the angle of the commissures of the valve, and thinning the wall of the vein near the valve sinuses. If dilation progresses sufficiently, the leaflets of the valve are unable to extend to one another, and consequently are unable to close the valve. All of the foregoing result in poor leaflet coaptation, and resulting valve reflux Secondary reflux usually follows thrombophlebitis, or inflammation in conjunction with the formation of a thrombus. Secondary reflux occurs where the valve is scarred, atrophic, thickened and deformed. Longitudinal septae may exist, along with a distorted lumen within the thickened vein wall.

Nonsurgical treatment of chronic venous insufficiency includes elevation of the legs, compression stockings, and, for venous ulcers, a boot made of rolled bandages containing a combination of calamine lotion, glycerin, zinc oxide and a gelatin. Traditional surgical approaches include vein ligation, axillary vein valve transfer, vein wrapping and valve repair through the precise placement of sutures internally or externally to the vein.

Implantable medical devices have been developed in recent years for the treatment of chronic venous insufficiency. Some devices act to mechanically constrict the vein circumferentially in order to reduce vein diameter. If a native valve has been rendered incompetent due to venous dilation, this approach is taken near the native valve in order to reestablish valve competence. Other devices have been developed to partially or totally flatten a vein in order to restore valve competence.

The foregoing surgical and non surgical approaches suffer numerous drawbacks as effective treatment for venous valve insufficiency. In addition to common post-operative complications such as wound hematoma, infection, lymphatic leak, and thrombosis, failure due to dilation, stenosis, distorted and thickened valve tissue, overly stretched leaflets, thin venous walls and other causes occur in a significant population of patients. Additionally, devices which narrow the vessel but do not repair valve leaflets may lead to increased redundancy, increased commisure angle, and be ineffective. An overly constricted vein may significantly reduce blood flow and potentially lead to vessel occlusion Similarly, difficulty in controlling lumen size and hemodynamic disruption in conjunction with a device designed to flatten a vein may lead to occlusion in a significant number of cases. Consequently, there remains a need in the art for an improved device for the treatment of venous valve insufficiency.

### SUMMARY OF THE INVENTION

An apparatus for improved functioning of a valve of a subject has a proximal region and a distal region, wherein the proximal region comprises an outward bias along a first axis and the distal region comprises an outward bias along a second axis. The first axis may be disposed at an angle of between approximately 45 degrees and approximately 135 degrees to the second axis. The proximal region may be placed at or near the commissures of a valve of a subject in order to improve the functioning of the valve. The proximal region may increase the distance between the commissures of a valve of a subject. The distal region may be configured to maintain patency of a lumen of a subject.

The apparatus may comprise a plurality of alternating peaks and valleys which may comprise a spring element and/or a height. The apparatus may comprise one or more legs joined by one or more peaks and valleys, and may also comprise one or more stabilizing elements disposed between the proximal region and the distal region. The peaks may be configured to maintain the patency of a lumen of a subject.

An alternative apparatus for the improved functioning of a valve of a subject may comprise a first and second leg separated by one or more peaks, the first and second leg comprising first and second shoulders which comprise a height. The peak may exert an outward bias on the first and second leg. The apparatus may be placed at or near a valve of a subject and to increase the distance between commissures of a valve of a subject.

Another apparatus for decreasing the distance between opposing leaflets of a valve of a subject may comprise a first arm and a second arm, and means for engaging opposing walls of a lumen of a subject. The first and second arms may comprise a bias toward one another and may be joined by one or more spring elements. The device may be generally linear or curvilinear. The apparatus is configured to penetrate opposing walls of a lumen of a subject, and comprises one or more means for limiting the depth to which said apparatus penetrates the opposing walls of a lumen of a subject. The device may further comprise a third and a fourth arm.

Yet another alternative apparatus for decreasing the distance between leaflets of a valve of a subject may have means for exerting a force primarily against the exterior of opposing walls of a lumen of a subject. Examples of such a device include one having a helical configuration, a mirror image first and second end, or a reverse mirror image first and second end.

Any of the foregoing embodiments may comprise shape memory materials, a delivery configuration and a deployed configuration, and means for engaging the walls of a lumen of a subject and/or for securing the device within a delivery system. Any may be implanted surgically, percutaneously, subcutaneously or other minimally invasive manner.

A method of improving the function of a valve of a subject is disclosed, where steps include implanting a device proximate a valve of a subject, wherein the device may be described as summarized above. A method may include the additional step or steps of compressing the vessel, removing a restraint from the device, expanding the device, or advancing the device. A delivery system having rails and means for expanding the device may be used.

### BRIEF DESCRIPTION OF THE DRAWINGS

FIG. 1 is a plan view of a schematic drawing of an incompetent venous valve.

FIG. 2 is a plan view of a schematic drawing of a venous valve following implantation of a device according to the invention.

FIG. 3 is a side view of an embodiment according to the invention.

FIG. 4 is a side view of an alternative embodiment according to the invention.

FIGS. 5-7 illustrate a cross-sectional side view of sequential steps in the deployment of an embodiment according to the invention.

FIG. 8 is a perspective view of an alternative embodiment according to the invention.

FIG. 9 is a perspective view of an alternative embodiment according to the invention

FIG. 10 is a perspective view of an alternative embodiment according to the invention.

FIG. 11 is a perspective view of an alternative embodiment according to the invention

FIG. 12 is a perspective view of an alternative embodiment according to the invention

FIG. 13 is a perspective view of an alternative embodiment according to the invention

FIG. 14 is a perspective view of an alternative embodiment according to the invention.

FIG. 15 is a perspective view of an alternative embodiment according to the invention

FIG. 16 is a cross-sectional frontal view of an incompetent valve within a vein.

FIG. 17 is a cross-sectional side view of the valve of FIG. 16.

FIG. 18 is a cross-sectional frontal view of the valve of FIG. 16 following treatment.

FIG. 19 is a cross-sectional frontal view of the valve of FIG. 17 following treatment.

FIG. 20 illustrates a side view of an embodiment according to the invention.

FIG. 21 illustrates a side view of an alternative embodiment according to the invention.

FIGS. 22-25 illustrate a cross-sectional side view of some of the steps of deployment of an embodiment according to the invention in a vessel.

FIG. 26 illustrates a side view of an embodiment according to the invention.

FIG. 27 illustrates a side view of an alternative embodiment according to the invention.

FIG. 28 illustrates a side view of an embodiment according to the invention.

FIG. 29 illustrates a side view of an embodiment according to the invention.

FIG. 30 illustrates a side view of an embodiment according to the invention.

FIGS. 31-35 illustrate a cross-sectional side view of some of the steps of deployment of an embodiment according to the invention.

FIG. 36 illustrates a side view of an embodiment according to the invention.

FIGS. 37-41 illustrate a cross-sectional side view of some of the steps of deployment of an embodiment according to the invention.

FIG. 42 is a side view of a deployment device (in its delivery configuration) for use in deployment of a device according to the invention.

FIG. 43 is the deployment device of FIG. 42 in a deployment configuration.

FIGS. 44-48 are cross-sectional side views illustrating sequential steps in the delivery and deployment of a device according to the invention utilizing the deployment device of FIGS. 42 and 43.

FIG. 49 is a side view of an alternative embodiment according to the invention.

FIG. 50 is a side view of an alternative embodiment according to the invention.

FIG. 51 is a side view of an alternative embodiment according to the invention.

FIG. 52 is a side view of an alternative embodiment according to the invention.

FIG. 53 is a side view of an alternative embodiment according to the invention.

FIG. 54 is a side view of an alternative embodiment according to the invention.

FIG. 55 is a side view of an alternative embodiment according to the invention.

FIG. 56 is a side view of an alternative embodiment according to the invention.

FIG. 57 is a side view of an alternative embodiment according to the invention.

FIG. 58 is a side view of an alternative embodiment according to the invention.

FIGS. 59-62 illustrate a cross-sectional side view of some of the steps of deployment of an embodiment according to the invention.

FIGS. 63-66 illustrate a cross-sectional side view of some of the steps of deployment of an embodiment according to the invention.

FIGS. 67-70 illustrate a cross-sectional side view of some of the steps of deployment of an embodiment according to the invention.

### DETAILED DESCRIPTION OF THE INVENTION

As utilized herein, the term “valvuloplasty” refers to the restoration of function of a valve, whether performed externally, internally, surgically, percutaneously, subcutaneously, mechanically, or through any combination of the foregoing.

As utilized herein, the term “expandable” refers to a device that comprises a reduced profile configuration and an expanded profile configuration. An expandable device may transition from a reduced profile configuration to an expanded profile configuration by mechanical means, by the application of an outward force, by self-expansion, or by any combination of the foregoing. The term “balloon expandable” refers to a device that comprises a reduced profile configuration and an expanded profile configuration, and may undergo a transition from the reduced configuration to the expanded configuration via the outward radial force of a balloon expanded by any suitable inflation medium. A “self-expanding” device has the ability to revert readily from a reduced profile configuration to a larger profile configuration in the absence of a restraint upon the device that maintains the device in the reduced profile configuration.

A device may be mechanically self-expanding and/or may be manufactured from a shape memory material. The term “balloon assisted” refers to a device the final deployment of which is facilitated by the expansion of or by utilization of an expanded balloon.

According to the inventions disclosed herein, a device is “implanted” if it is placed within the body to remain for any length of time following the conclusion of the procedure to place the device within the body. A device according to the invention may be manufactured from a suitable biocompatible metal such as, for example surgical stainless steel, nickel titanium alloy (or “nitinol”), CoCr alloy, MP35N, Mg, Ag, gold, and others. A device according to the invention may alternatively be manufactured from a suitable polymer such as polyurethane, nylon, polyethylene terephthalate, polyester, polyethylene, polypropylene, and others.

“Shape memory” refers to the ability of a material to undergo structural phase transformation such that the material may define a first configuration under particular physical and/or chemical conditions, and to revert to an alternate configuration upon a change in those conditions. Shape memory materials may be metal alloys including but not limited to nickel titanium, or may be polymeric.

Any of the devices described below may comprise radiopaque markers in order to enhance visualization of the device under fluoroscopy. Examples of suitable radiopaque markers include, but are not limited to Gold or Platinum bands or markers, tantalum, bismuth oxide, barium sulfide and others.

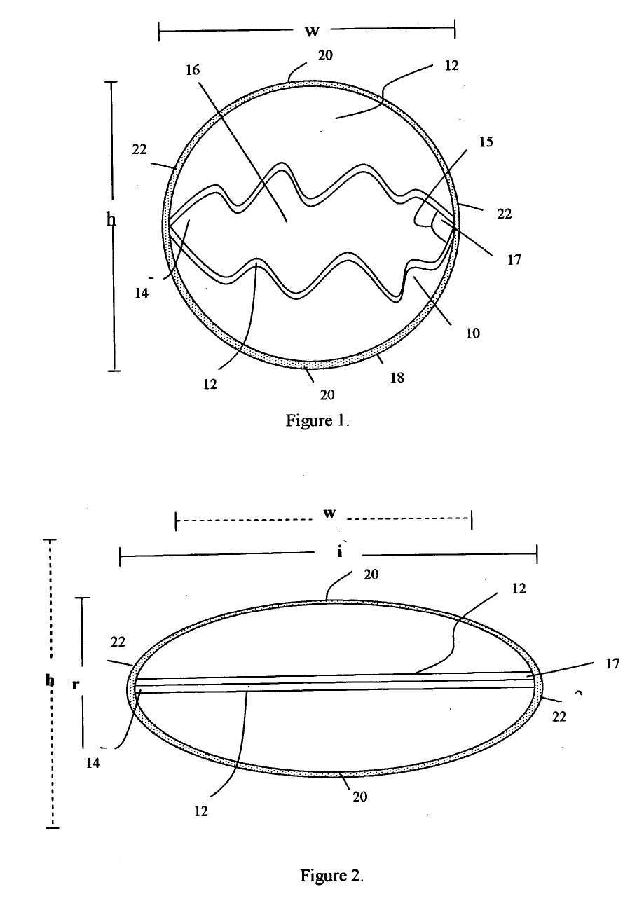

A venous valve having diminished competence can be characterized as in FIG. 1, which is a schematic representation of such a valve from a plan view, or looking down onto the top of the valve from within the lumen. (The remainder of the vein is not pictured.) Valve 10, shown in FIG. 1, is dilated, and comprises leaflets 12, commissures 14 and wall 18. Opposing sides 20 and 22 of walls 18 are separated by first distances h and w respectively. Because valve 10 is somewhat dilated, and because leaflets 12 are overstretched, redundant, and of irregular morphology, wall 18 is somewhat thinned at commissures 14, and valve 10 continually comprises commissure angles 15 and valve opening 16. Valve leaflets 12 are unable to coapt, and valve 10, instead of alternating between a “closed” and “open” position, remains in the generally “open” configuration illustrated in FIG. 1.

Turning now to FIG. 2, a plan view of the same valve within the vein, following implantation of a device according to the invention (not pictured), is illustrated. A device according to the invention (not pictured) has been implanted in the interior of the vessel of valve 10 either proximal to or distal to, or both proximal and distal to valve 10. First distance w has increased to become increased distance i. First distance h between opposing walls 20 has been reduced to second distance r. Overstretched and redundant leaflets 12 are thereby extended along their length between commissures 14 and 17 and are now able to coapt when valve 10 is closed. Further, when valve 10 is closed, commissure angles 15, prolapse and valve opening 16 are eliminated or nearly completely eliminated. Competence of valve 10 is thereby restored, preventing reflux and further swelling of the vessel.

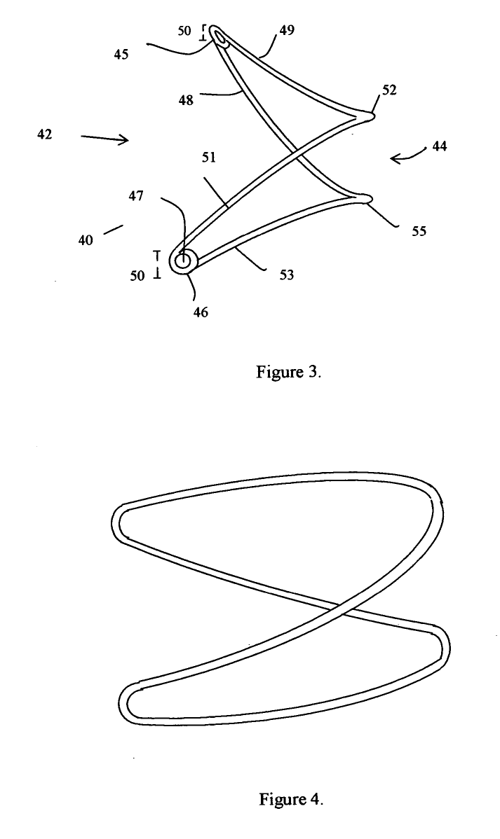

FIG. 3 is an example of a device according to the invention which may be implanted in order to achieve the repair of a venous valve as set forth above. Although numerous variations are possible within the scope of the invention, valvuloplasty device 40 comprises proximal end 42 which, when implanted, is placed proximal to a venous valve (not pictured.) Device 40 further comprises distal end 44 which, in use, is placed distally of a venous valve. Such a device may be delivered and deployed percutaneously utilizing a catheter or comparable delivery system, or may be implanted surgically.

Device 40 generally comprises a substantially continuous wire, filament or other elongated piece of material configured to comprise opposing legs 48 and 49 which substantially converge at proximal peak 45. (Alternatively, device 40 may be comprised of separate filaments joined together.) Device 40 also comprises opposing legs 51 and 53 which substantially converge at proximal peak 46. Proximal peaks 45 and 46, in the embodiment of FIG. 3, comprise loops 47. (Other embodiments may not comprise loops 47. Such devices, however, remain within the scope of the invention.) Loops 47 may enhance the spring action of proximal peaks 45 and 46 which biases opposing legs 48 and 49 apart, and 51 and 53 apart, and may increase the height of proximal shoulders 50. During deployment of a device such as device 40, proximal peaks 45 and 46 are placed in proximity to the commissures of a venous valve (not pictured). Device 40 may be self expanding, balloon expandable, mechanically expandable, or a combination of the foregoing.

Opposing legs 49 and 51 extend to form distal peak 52 at distal end 44. Similarly, opposing legs 48 and 53 substantially converge to define distal peak 55. The spring bias within distal peaks 52 and 55 forces proximal peaks 45 and 46 apart. Congruently, the spring bias of proximal peaks 45 and 46 forces distal peaks 52 and 55 apart, but in a direction perpendicular to the bias of proximal peaks 45 and 46. Consequently, proximal peaks 45 and 46, placed in proximity to the commissures of a venous valve undergoing treatment (not pictured) force the commissures apart from one another, increasing the distance between commissures, and decreasing the distance between opposing walls of the vessel which are perpendicular to the walls which define the commissures, as described in relation to FIG. 1 above. Increasing the distance between commissures and shortening the distance between opposite walls in a direction perpendicular to the line where the leaflets meet to close the valve, make it easier for the leaflets to meet. Further, the loose, redundant leaflets are tightened, and the commissure angle and prolapse is reduced.

Further, upon deployment of device 40, distal peaks 52 and 55 are biased away from one another up against and/or into the walls of the vessel (not pictured) in a direction perpendicular to the direction peaks 45 and 46 are biased apart. Consequently, while peaks 45 and 46 increase the distance between commissures of the valve undergoing treatment, (and decrease the distance between the walls perpendicular to the walls forming the commissures), distal peaks 52 and 55 support a continued distance between these opposing walls of the vessel, thereby preventing occlusion of the vessel. In other words, the device acts to mechanically remodel the vein, reestablishing valve competence without compromising lumen area Distal peaks 52 and 55 also serve to securely anchor device 40 within the vessel.

In other embodiments, the device may comprise varied configurations, including, but not limited to more rounded peaks, fewer or more loops, additional features for attachment to the vessel wall, and others. An example of an alternative device is set forth in FIG. 4. Examples of features for attachment to the vessel wall include, but are not limited to, one or more projections, barbs, umbrella connectors, or other suitable means. Fixation of any of the foregoing or other attachment means may be facilitated by a balloon, a mechanical expansion device, or may occur as a result of the self-expanding nature of the device.

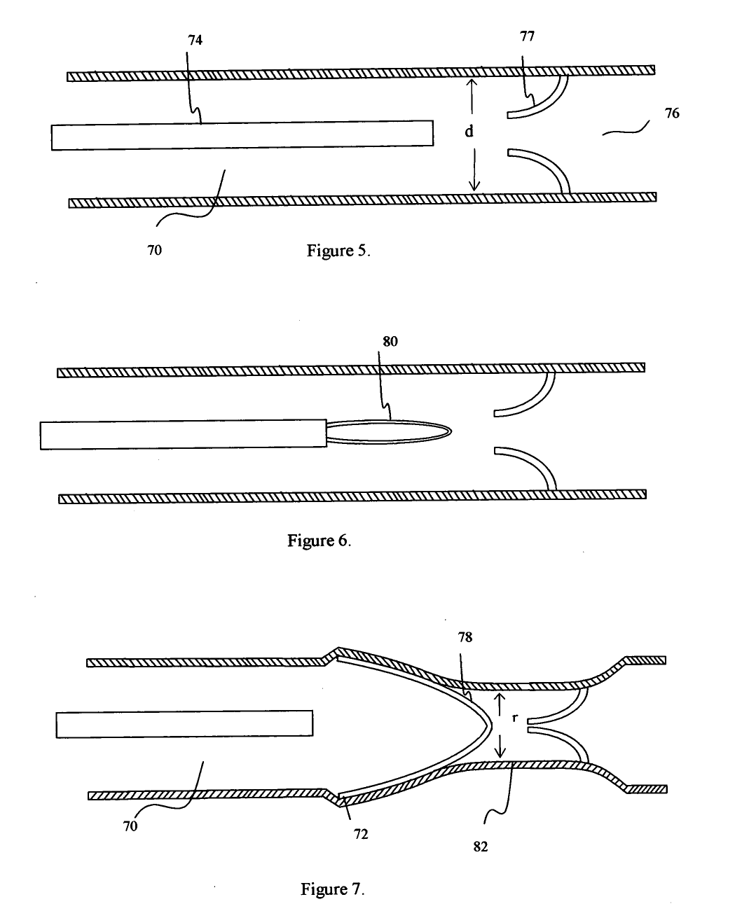

FIGS. 5-7 illustrate some of the steps taken during deployment of a device similar to that described in relation to FIGS. 3 and 4 above. FIGS. 5-7 illustrate a cross-sectional side view of vessel 70 into which delivery catheter 74 has been introduced and positioned proximate damaged valve 76. Damaged valve 76 comprises leaflets 77 which are unable to coapt, leaving damaged valve 76 in a perpetually “open” position. Valvuloplasty device 80, in its delivery configuration, is carried within delivery catheter 74. Once delivery catheter 74 has been properly positioned within vessel 70, valvuloplasty device 80 is ejected (or delivery catheter 74 is withdrawn over device 80). Device 80 is thereby permitted to achieve its deployed configuration through spring or other mechanical action, or through the material's shape memory properties. Valvuloplasty device 80, seen in cross section and therefore in only one plane in FIG. 7, is biased against wall of vessel 70 at its distal end 72, distal to valve 76. Proximate valve 76, proximal end 78 of device 80 is biased apart in a plane perpendicular to the plane of expansion of distal end 72. The expansion of proximal end 78 along the direction of the line where leaflets 77 meet results in an increased distance between commissures, thereby stretching leaflets 77 along the line where they meet (not pictured). Further, expansion of proximal region 78 along the line where leaflets 77 meet results in a decreased distance r across vessel 70. As a result of the reduced distance r, leaflets 77 are able to meet, and valve 76 is now able to close. As suggested above, a device may be placed on either or both sides of valve 76.

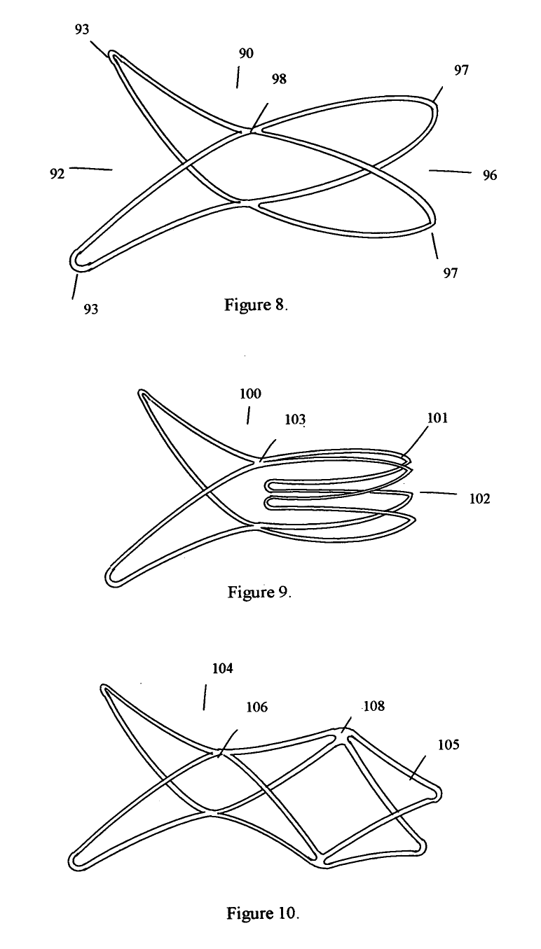

FIGS. 8-10 illustrate alternative embodiments according to the invention which function in much the same manner as the examples set forth above. Valvuloplasty device 90, illustrated in FIG. 8, comprises proximal portion 92 comprising an outward spring bias in a first direction, and distal portion 96 comprising an outward spring bias in a direction generally close to that of the first direction of bias. The distance between proximal peaks 93 may be slightly greater than the distance between distal peaks 97. Other configurations, such as, for example, alternative angles between legs, more angular and less curvilinear geometries, wider or more narrow flaring between peaks, convexity or concavity of regions, barbs or other suitable vessel wall attachment means, and other variations are possible within the scope of the invention. Further, device 90 may comprise a unitary piece or may be constructed by linking two or more portions to form the device. Device 90 comprises integral region or regions 98. Integral regions 98 may comprise a region where the legs of the device are integral with one another or are linked to one another, such as by clamping, welding, sintering, melting, or other suitable means.

FIGS. 9-10 set forth additional examples of devices according to the invention which comprise features similar to those discussed above in relation of FIG. 8. Device 100 of FIG. 9 comprises multiple peaks 101 disposed about distal region 102. Peaks 101 confer additional stability on device 100 when it is anchored in vivo, typically within the dynamic environment of a relatively elastic blood vessel through which the device is subjected to vessel movement and blood flow. Similarly, crown 105 illustrated in FIG. 10 confers additional stability upon device 104. Devices 100 and 104 both comprise integral regions 103, 106 and 108. As set forth above in relation to the descriptions of alternative embodiments, numerous iterations of the foregoing embodiments are possible within the scope of the invention set forth herein.

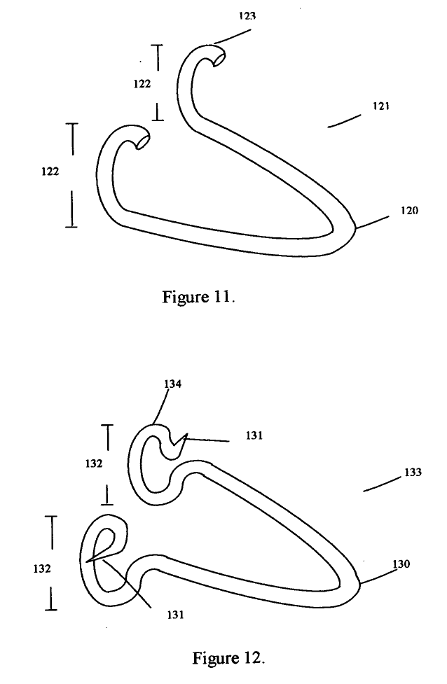

Alternative embodiments are illustrated in FIGS. 11-12. Both device 121 of FIG. 11 and device 133 of FIG. 12 function to increase the distance between commissures of a valve when implanted in a vessel (not pictured). In use, shoulders 123 (or 134 in the embodiment illustrated in FIG. 12) are seated at or near the commissures of a valve (not pictured). The spring action of apex 120 (or apex 130) biases shoulders 123 (or shoulders 134) apart, thereby increasing the distance between valve commissures, stretching the leaflets and improving coaptation of the leaflets in much the same manner as discussed above in relation to alternative embodiments. Further, height 122 of shoulders 123 and height 132 of shoulders 134 function to prevent the lumen from closing completely, thereby maintaining fluid flow therethrough In addition, barbs 131 of device 133 further secure device 133 once implanted in a vessel. Other securing means such as those set forth as examples above may be used alternatively or in addition to barbs 131.

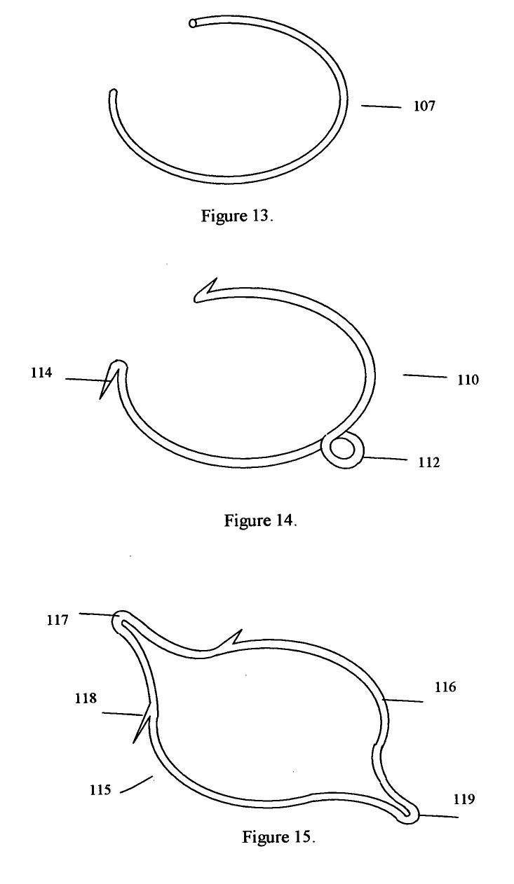

Turning now to FIGS. 13-19, alternative embodiments according to the invention and examples of steps of deployment of such embodiments are described. Shown in its simplest form in FIG. 13, valvuloplasty device 107 comprises a generally incompletely circular device. As shown in FIGS. 14 and 15, valvuloplasty devices 110 and 115 comprise a comparable configuration with some additional features. Valvuloplasty device 110, for example, comprises an incompletely circular configuration, spring loop 112 and barbs 114. Valvuloplasty device 115, on the other hand, comprises a generally circular portion 116, proximal peak 117, barbs 118, and distal peak 119. As with all examples set forth herein, numerous other configurations, spring means, attachment means, and geometries are possible within the scope of the invention.

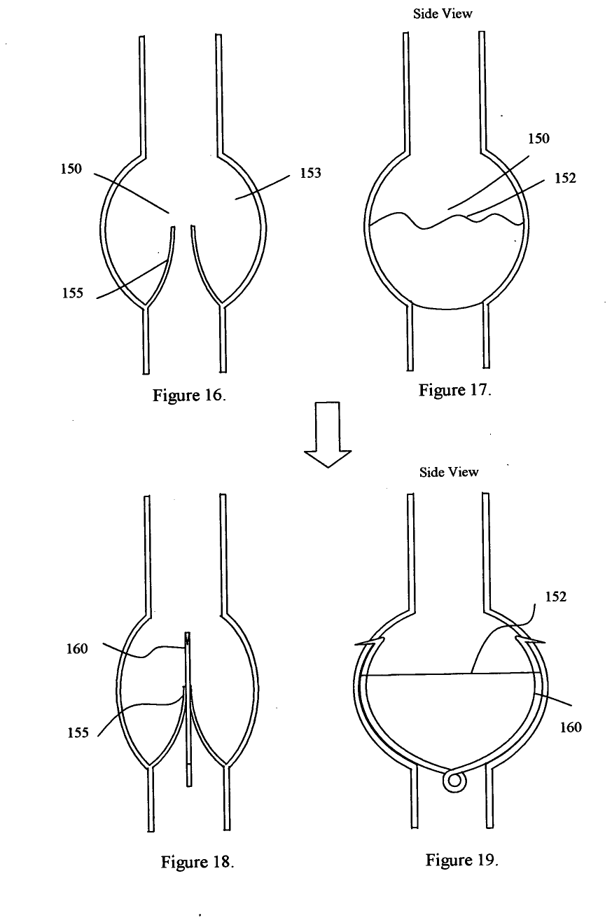

FIGS. 16-19 illustrate cross-sectional views of valve 150 before and after deployment of a device similar to those discussed in relation to FIGS. 13-15. FIG. 16 illustrates a cross-sectional view of valve 150 before treatment, taken perpendicular to the line where leaflets 155 meet (or would meet in a healthy valve). Leaflets 155 do not meet in FIG. 16, as they are damaged, or vessel 153 is overly dilated, or both FIG. 17 illustrates a side view of valve 150 in a plane perpendicular to that of the previous figure, also before treatment. Leaflet attachment line 152 is characterized by an irregular geometry to represent unhealthy, stretched leaflets. FIGS. 18 and 19 illustrate congruent views to those of FIGS. 16 and 17 respectively, following deployment of device 160. Device 160 is deployed while seated substantially within valve 150, increasing the distance between the commissures (not pictured) of valve 150, tightening and allowing leaflets 155 to meet, thereby restoring function of valve 150.

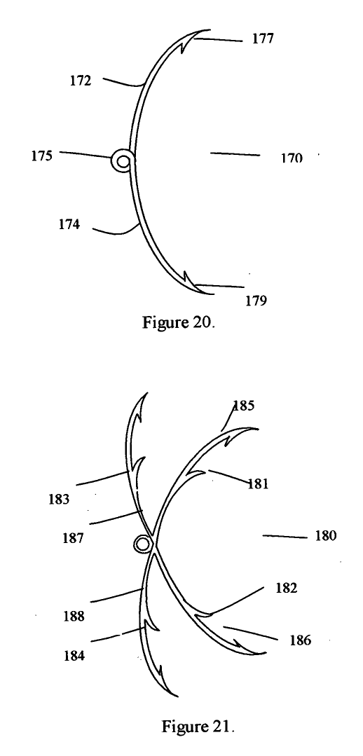

In an alternative approach to treating venous valve insufficiency, the devices and methods illustrated in FIGS. 20-70 function to narrow the distance between the walls of a vein proximate a valve in order to restore function to the valve. FIG. 20 illustrates a side view of valvuloplasty device 170 comprising first and second arms 172 and 174, spring element 175, and barbs 177 and 179. Numerous alternative configurations of spring element 175 are suitable according to the invention Further, additional barbs and alternative configurations of barbs 177 and 179 fall within the scope of the invention. Still further, multiple devices such as device 170 may be used together and/or may be linked to one another, as illustrated in FIG. 21.

Device 180, in addition to comprising multiple devices, also comprises optional penetration stoppers 181, 182, 183 and 184. Penetration stoppers 181, 182, 183, 184 function to limit the penetration depth of arms 185, 186, 187, 188 when implanted in a vessel in a subject. Examples such as device 170 or devices similar thereto could also comprise penetration stoppers in alternative embodiments.

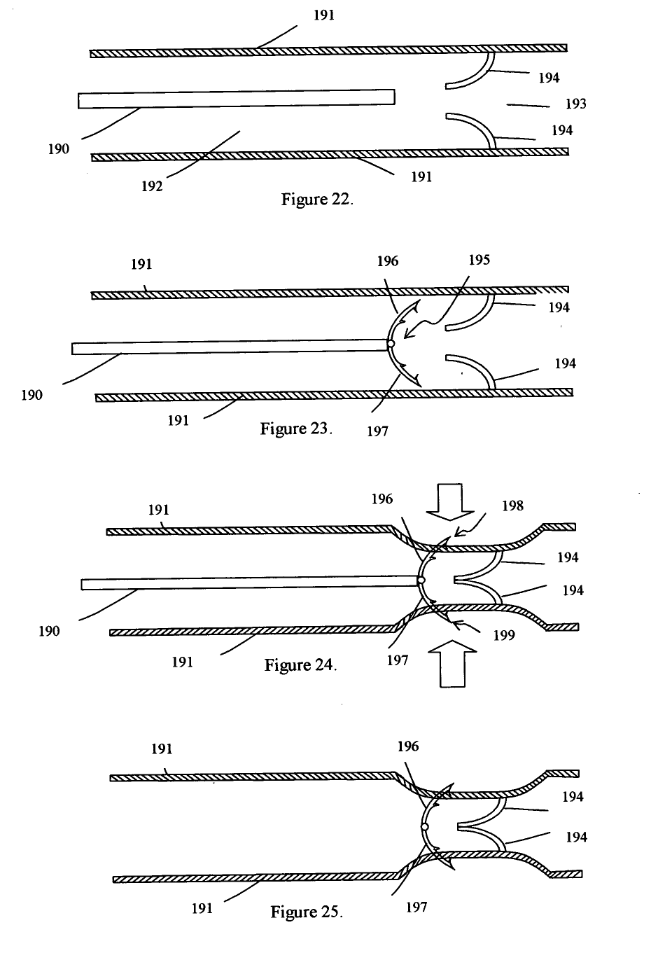

Some sequential steps of deployment of a device similar to devices 170 or 180 are illustrated in FIGS. 22-25. In a preliminary step, introducer 190 is placed percutaneously within vessel 192, proximate valve 193. Once proper positioning is confirmed via fluoroscopy or ultrasound, device 195 is forced out of introducer 190. However, device 195 is still attached to the introducer 190 through a suture, wire, cable or other attachment means. A suitable means of external compression (not pictured) of vessel 192 is then employed in order to compress vessel 192 and to engage the walls of vessel 192 and arms 196 and 197. An example of a suitable means of compression is a compression cuff similar to that used in measuring blood pressure. Arms 196 and 197 of device 195 penetrate vessel walls 191, and barbs 198 and 199 secure the engagement, preventing withdrawal of arms 196 and 197 from vessel walls 191. Engagement of walls 191 by device 195 brings opposing walls 191 closer to one another, thereby reducing the distance between the leaflets 194. As the distance between the leaflets 194 is reduced, leaflets 194 of valve 193 are permitted to meet and coapt, thereby restoring function of valve 193. The attachment means is then removed to release the device 195.

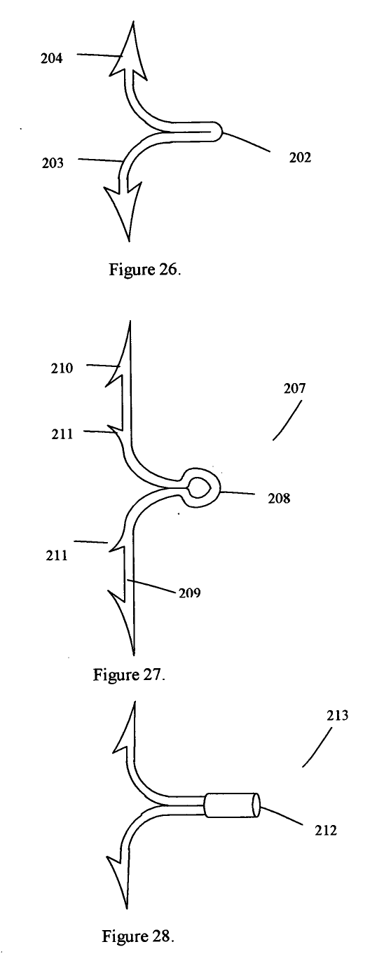

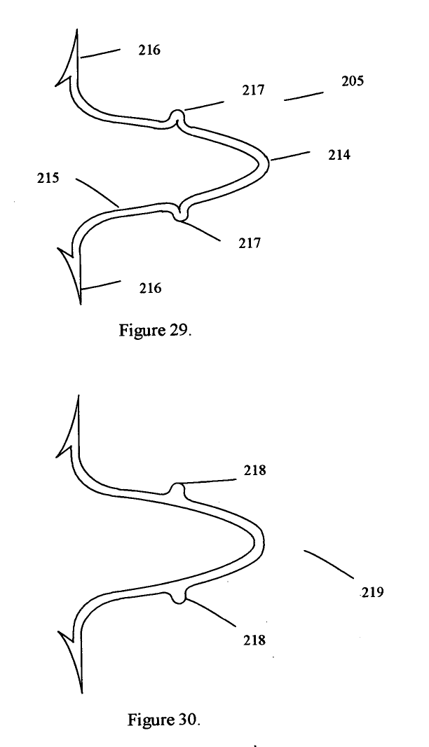

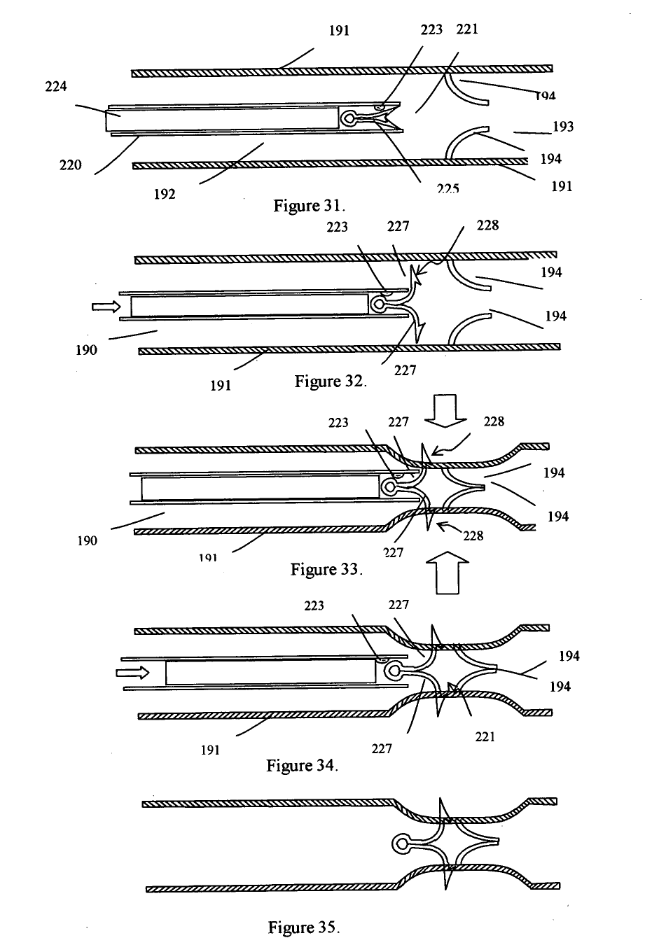

FIGS. 26-30 illustrate additional examples of embodiments according to the invention which function to reduce the distance between the leaflets of a vessel in order to restore valve function. Numerous other configurations of spring elements 175, 202, 208, 212 and 214 are suitable according to the invention Similarly, numerous alternative configurations of barbs 177, 179, 204, 210 and 216, and arms 172, 174, 203, 209 and 215 are possible. Further, as illustrated in FIGS. 29 and 30, devices 205 and 219 comprise optional deployment stoppers 217 and 218 respectively which are used to secure the devices in the delivery system before final placement in the vessel. Comparable features are described more fully below in relation to FIGS. 31-35. Alternatively, or in addition, spring elements 208 and 212 of devices 207 and 213 serve to secure the respective devices within a delivery system during placement of the delivery system within a vessel. Alternatively, a suture or a wire in the delivery system can be used to secure the device in the catheter before final placement of the system in a vessel.

FIGS. 31-35 illustrate some sequential steps in the deployment of a device similar to the devices illustrated in FIGS. 26-30 above. In FIG. 31, introducer 220 having pusher rod 224 and carrying device 225 is positioned proximate a non-functioning valve 193. Following positioning, pusher rod 224 partially expels device 225, allowing arms 227 to transition to a deployment configuration. Bump stop 223 secures device 225 within the distal end of introducer 220 at this stage in deployment and prevents final expulsion of device 225 from introducer 220. While device 225 is secure and arms 227 are in a deployment configuration, an external means of compression not pictured may be applied in order to engage arms 227 and the walls of a vessel 191. Barbs 228 secure arms 227 within the walls of such a vessel. Following compression and engagement, pusher rod 224 can again be utilized in order to expel device 225 beyond bump stop 223 and from distal end 221 of introducer 220. Introducer 220 can then be removed and device 225 can be left in place in the vessel.

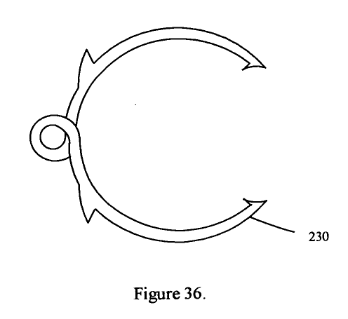

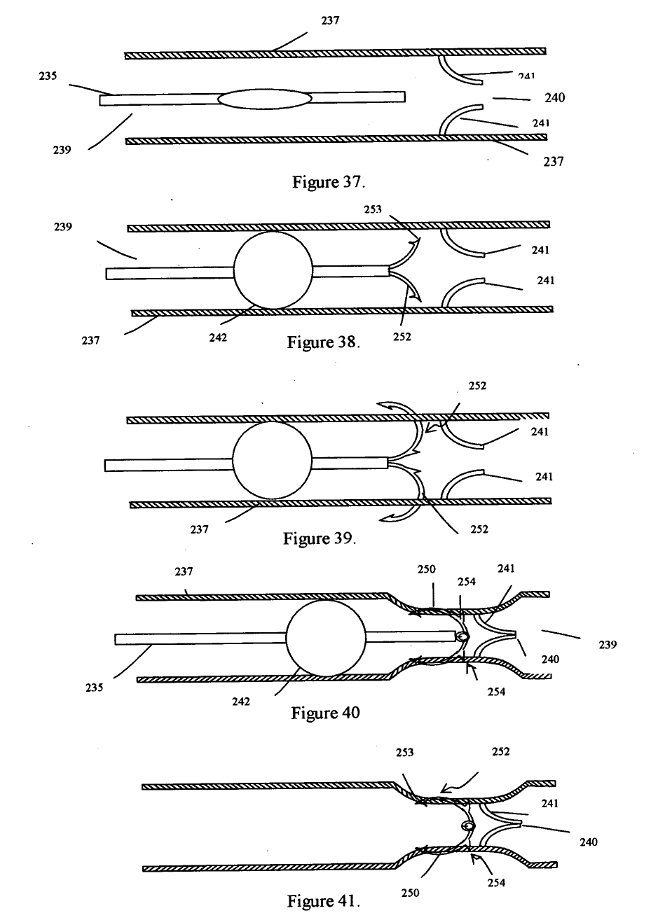

FIG. 36 illustrates an alternative embodiment according to the invention. Numerous alternative configurations of device 230 and its features are suitable according to the invention FIGS. 37-40 represent a cross-sectional side view of some of the sequential steps used in order to deploy a device such as device 230. As illustrated in FIG. 37, introducer 235 is percutaneously introduced into vessel 239 and placed near non-functioning valve 240 comprising leaflets 241. Balloon 242 or other suitable securing means is inflated in order to secure introducer 235 within vessel 237. Device 250 comprising arms 252 and barbs 253 is gradually expelled from introducer 235. As illustrated in FIG. 39, as expulsion of device 250 continues, arms 252 transition to a deployment configuration, piercing vessel walls 237 from the interior as they transition Arms 252 again pierce vessel walls 237 from the exterior of vessel 239 as they continue to transition to a deployment configuration, as illustrated in FIG. 40. Barbs 253 secure arms 252 and device 250 after device 250 is completely expelled from introducer 235. Optionally, spacers 254 are used to determine the penetration depth of arms 252 and the distance between the vessel wall 237. Further, device 250 engages walls 237 and decreases the distance between the leaflets of vessel 239. As a result, valve leaflets 241 are brought closer to one another, are permitted to coapt, and function of valve 240 is thereby restored. Thereafter, balloon 242 is deflated, and introducer 235 is removed, leaving device 250 within vessel 239. Multiple devices such as device 250 may be deployed near a valve, on either or both sides of the valve within the vessel.

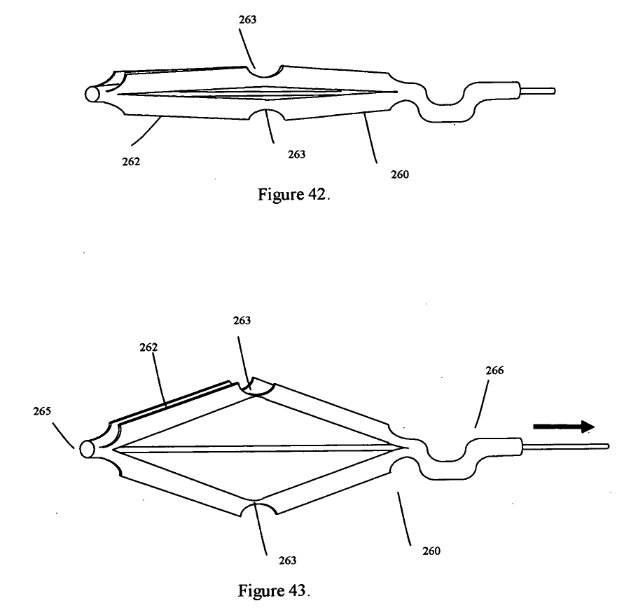

Turning now to FIGS. 42-48, an alternative method of deployment and an alternative deployment device according to the invention are illustrated. FIG. 42 illustrates a side view of an alternative deployment device 260 in or near its low-profile delivery configuration. In its delivery configuration, deployment scaffolds 262 of deployment device 260 are substantially linear. While undergoing deployment, deployment scaffolds 262 “buckle”, or bend at scaffold hinges 263 in order to transition from a substantially linear configuration to define two substantially ‘V’-shaped configuration. Scaffold hinge 263 may be of any configuration suitable to allow scaffold 262 to bend. Further, scaffold 262 may be subjected to any force suitable to cause scaffold 262 to bend, including, for example, a pulling back of distal end 265, a pushing forward of proximal end 266, or both, or other suitable action. FIG. 43 illustrates deployment device 260 in or near its deployment configuration.

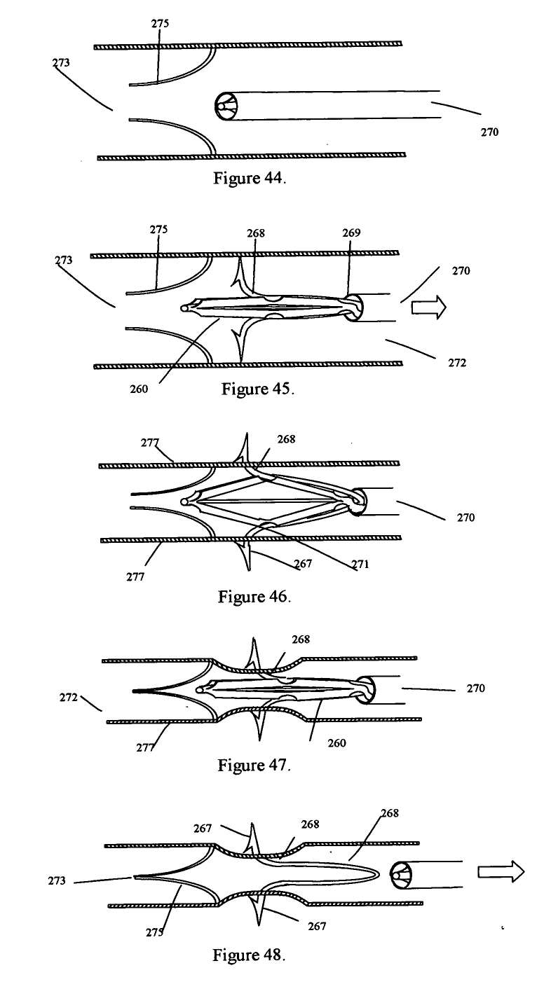

FIGS. 44-48 illustrate sequential steps of deployment of a valvuloplasty device 268 using deployment device 260. In FIG. 44, introducer 270 has been percutaneously placed within vessel 272 near non-functioning valve 273. Deployment device 260, with valvuloplasty device 268 mounted thereon, is emerging from distal end 269 of introducer 270. Valve leaflets 275 are unable to coapt in valve 273. Deployment device 260 is placed in its deployment configuration as illustrated in FIG. 46, and arms 268 are forced outwardly to engage vessel wall 277. Barbs 267 secure the engagement of arms 271 and vessel walls 277.

As illustrated in FIG. 47, following engagement of valvuloplasty device 268 and vessel walls 277, deployment device 260 is returned to its delivery configuration, and valvuloplasty device 268 transitions to its final deployment configuration due to the elastic property of the device 268. As device 268 transitions to its final deployment configuration, it pulls the walls 277 more closely together. Deployment device 260 is withdrawn into introducer 270, and both are removed from vessel 272, leaving valvuloplasty device 268 implanted. Valvuloplasty device 268 pulls walls 277 more closely together, allowing valve leaflets 275 to coapt, and thereby restoring function to valve 273.

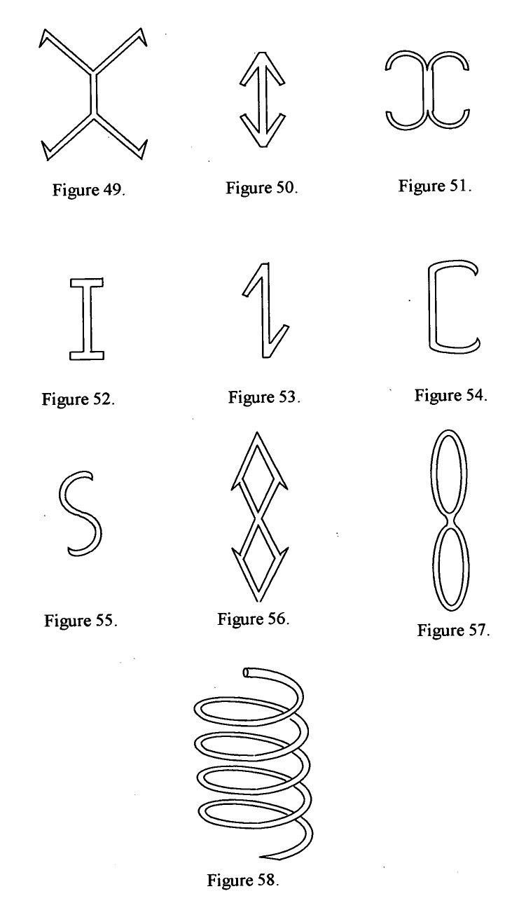

FIGS. 49-46 illustrate several examples of alternative embodiments according to the invention which are designed to reduce the distance between the leaflets of an overly dilated vein, and some of the steps followed to deliver and deploy such devices from the exterior of a vein. FIGS. 49-58 are side views of several examples of such devices. Numerous other iterations of devices and additional features such as, for example, barbs, are also within the scope of the invention.

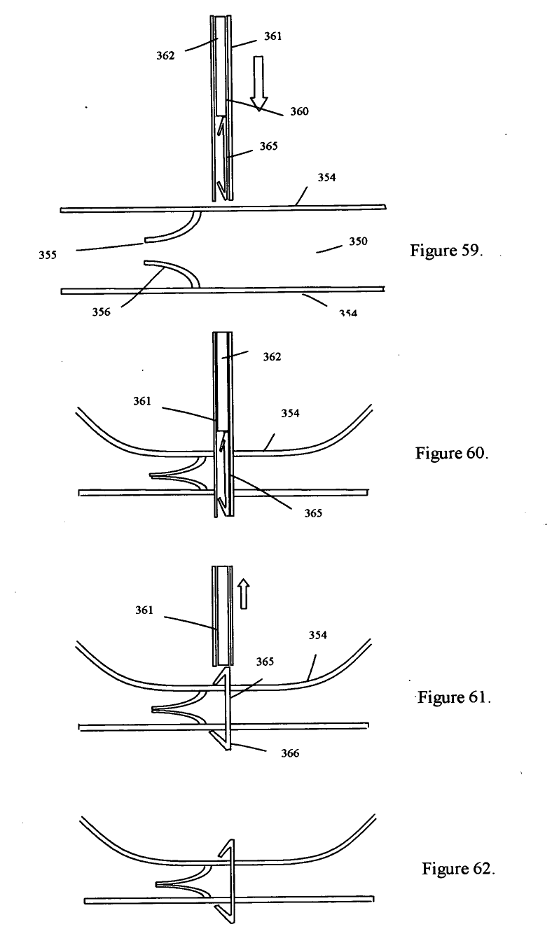

FIGS. 59-62 illustrate, in cross-sectional side view, sequential steps in the delivery and deployment of a valvuloplasty device similar to those set forth in FIGS. 49-58. FIG. 59 depicts vessel 350, having walls 354, non-functional valve 355 having valve leaflets 356. Introducer 360 having outer sheath 361 and pusher rod 362 and carrying valvuloplasty device 365 is also illustrated. In FIG. 60, introducer 360 is shown following penetration of vessel 350 proximate valve 355. As introducer penetrates vessel 350, walls 354 are forced closer to one another. Valvuloplasty device 365 is ejected from introducer 360 by retraction of outer sheath 361 while pusher rod 362 remains in place and transitions to a deployment configuration in FIG. 61. Arms 366 secure valvuloplasty device 365 in engagement with walls 354, and introducer 360 is removed. As a result of implantation of valvuloplasty device 365, leaflets 356 are able to coapt, and function of valve 355 is restored.

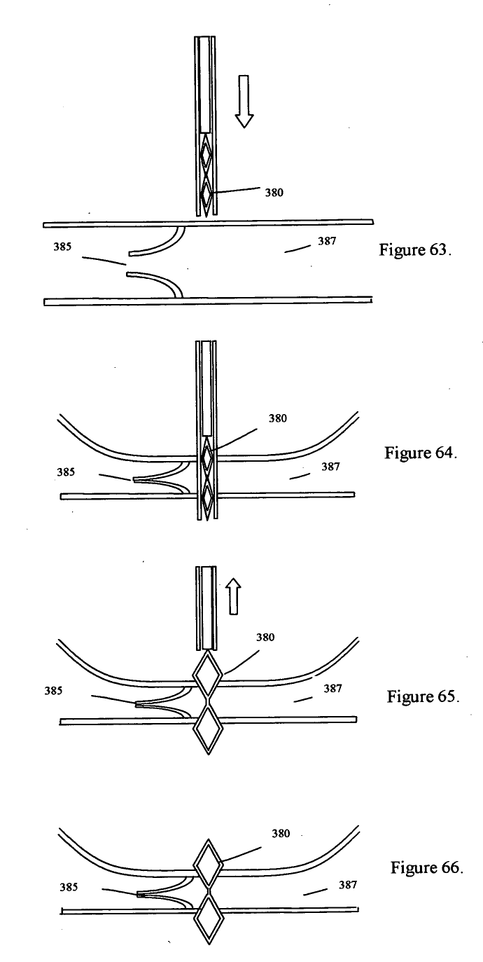

FIGS. 63-66 illustrate similar sequential steps in the introduction and deployment of valvuloplasty device 380 near valve 385 in vein 387.

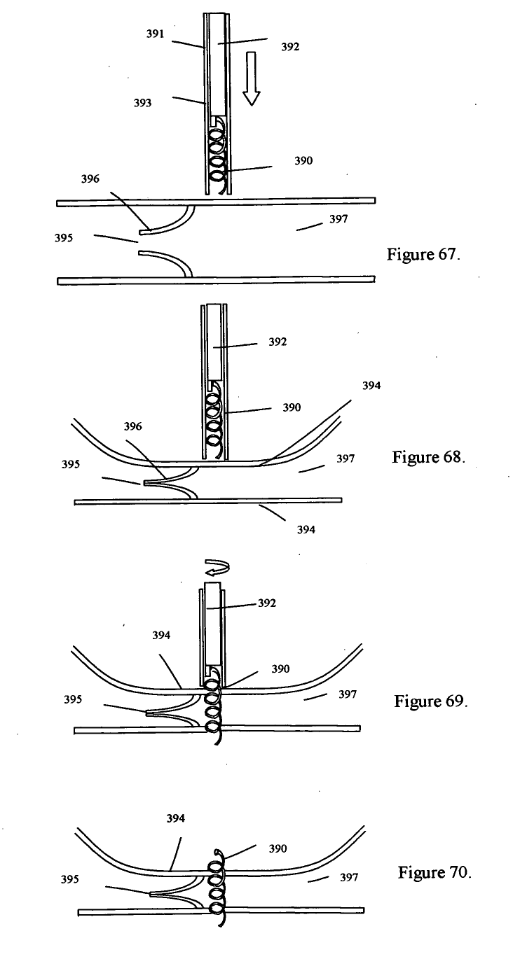

FIGS. 67-70 illustrate sequential steps in the introduction and deployment of valvuloplasty device 390 near valve 395 in vein 397. Introducer 391 is first positioned near valve 395 outside vein 397. Torque rod 392, in communication with generally helical device 390, is rotated. Rotation of torque rod 392 thereby exerts a rotational force upon device 390. Device 390 thereby advances into the interior of vein 397 until it penetrates and engages opposing walls 394 of vein 397. Absent a rotational force, device 390 remains in place in opposing walls 394, maintaining leaflets 396 in coaptation, thereby restoring function of valve 395.

While particular forms of the invention have been illustrated and described above, the foregoing descriptions are intended as examples, and to one skilled in the art it will be apparent that various modifications can be made without departing from the spirit and scope of the invention

I claim:

1. An apparatus for improved functioning of a valve of a subject, said apparatus comprising a proximal region and a distal region, wherein said proximal region comprises an outward bias along a first axis and said distal region comprises an outward bias along a second axis.

2. The apparatus according to claim 1, wherein said first axis is disposed at an angle to said second axis.

3. The apparatus according to claim 2, wherein said angle is between approximately 45 degrees and approximately 135 degrees.

4. The apparatus according to claim 1, wherein said proximal region is configured to be placed at or near the commissures of a valve of a subject.

5. The apparatus according to claim 1, wherein said proximal region increases the distance between the commissures of a valve of a subject when implanted in a lumen of a subject.

6. The apparatus according to claim 1, wherein said distal region is configured to maintain patency of a lumen of a subject.

7. The apparatus according to claim 1 wherein said apparatus comprises a plurality of alternating peaks and valleys.

8. The apparatus according to claim 1 wherein one or more of said peaks or one or more of said valleys comprises a spring element.

9. The apparatus according to claim 7 wherein one or more of said peaks or one or more of said valleys comprises a height.

10. The apparatus according to claim 1 wherein said apparatus comprises one or more legs joined by one or more peaks and valleys.

11. The apparatus according to claim 1 wherein said apparatus comprises one or more stabilizing elements.

12. The apparatus according to claim 11 wherein one or more of said stabilizing elements is disposed between said proximal region and said distal region.

13. The apparatus according to claim 12 wherein said distal region comprises a plurality of peaks and valleys.

14. The apparatus according to claim 13 wherein said distal region comprises one or more stabilizing elements.

15. The apparatus according to claim 1 further comprising one or more means for engaging the walls of a lumen of a subject.

16. The apparatus according to claim 1 wherein said apparatus comprises one or more shape memory materials.

17. The apparatus according to claim 10 wherein one or more of said peaks is configured to maintain the patency of a lumen of a subject.

18. An apparatus for the improved functioning of a valve of a subject, said apparatus comprising a first and second leg separated by one or more peaks, said first and second leg comprising first and second shoulders.

19. The apparatus according to claim 18 wherein said peak exerts an outward bias on said first and second leg.

20. The apparatus according to claim 18 wherein said first shoulder comprises a height and said second shoulder comprises a height.

21. The apparatus according to claim 18 wherein one or more of said legs comprises one or more means for engaging the walls of a lumen of a subject.

22. The apparatus according to claim 18 wherein said apparatus is configured to be placed at or near a valve of a subject.

23. The apparatus according to claim 22 wherein said apparatus is configured to increase the distance between commissures of a valve of a subject.

24. The apparatus according to claim 18 wherein said first and second shoulders are configured to maintain the patency of a lumen of a subject.

25. The apparatus according to claim 18 wherein said apparatus comprises one or more shape memory materials.

26. An apparatus for decreasing the distance between opposing leaflets of a valve of a subject, said apparatus comprising a first arm and a second arm, said first and second arm comprising means for engaging opposing walls of a lumen of a subject.

27. The apparatus according to claim 26 wherein said first and second arms comprise a bias toward one another.

28. The apparatus according to claim 26 wherein said first and second arms are joined by one or more spring elements.

29. The apparatus according to claim 26 wherein said one or more means for engaging opposing walls of a lumen of a subject comprises one or more barbs.

30. The apparatus according to claim 26 wherein said apparatus is configured to penetrate opposing walls of a lumen of a subject, and said apparatus comprises one or more means for limiting the depth to which said apparatus penetrates the opposing walls of a lumen of a subject.

31. The apparatus according to claim 26 wherein said apparatus further comprises a third and a fourth arm.

32. The apparatus according to claim 26 wherein said apparatus further comprises means for securing the apparatus within a delivery system.

33. The apparatus according to claim 32 wherein said means for securing the apparatus comprises one or more protrusions.

34. The apparatus according to claim 33 wherein said one or more protrusions is configured to engage the interior of a delivery system.

35. The apparatus according to claim 26 wherein said apparatus comprises a delivery profile, an expanded profile, and a deployed profile.

36. The apparatus according to claim 35 wherein said expanded profile is greater than said deployed profile.

37. The apparatus according to claim 26 wherein said apparatus is delivered percutaneously.

38. The apparatus according to claim 26 wherein said apparatus is delivered subcutaneously.

39. An apparatus for decreasing the distance between leaflets of a valve of a subject, said apparatus comprising means for exerting a force primarily against the exterior of opposing walls of a lumen of a subject, wherein said device may be delivered subcutaneously.

40. The apparatus according to claim 1 wherein said apparatus comprises a delivery configuration and a deployed configuration.

41. The apparatus according to claim 18 wherein said apparatus comprises a delivery configuration and a deployed configuration.

42. The apparatus according to claim 26 wherein said apparatus comprises a delivery configuration and a deployed configuration.

43. A method of improving the function of a valve of a subject, said method comprising the step of:
implanting a device proximate a valve of a subject, wherein said device comprises a proximal region and a distal region, wherein said proximal region comprises an outward bias along a first axis and said distal region comprises an outward bias along a second axis.

44. The method according to claim 43 wherein said device is implanted in a minimally invasive manner.

45. The method according to claim 43 wherein said device comprises a delivery configuration and a deployed configuration, and said step of implanting the device comprises the steps of percutaneously delivering the device and deploying the device.

46. The method according to claim 45 wherein said step of percutaneously delivering the device comprises the step of constraining the device within a delivery device, positioning the device proximate a valve; and wherein
the step of deploying the device comprises the step of withdrawing the constraint.

47. A method of improving the function of a valve of a subject, said method comprising the step of:
implanting a device proximate a valve of a subject, wherein said device comprises a first and second leg separated by one or more peaks, said first and second leg comprising first and second shoulders.

48. The method according to claim 47 wherein said device is implanted in a minimally invasive manner.

49. The method according to claim 47 wherein said device comprises a delivery configuration and a deployed configuration, and said step of implanting the device comprises the steps of percutaneously delivering the device and deploying the device.

50. The method according to claim 49 wherein said step of percutaneously delivering the device comprises the step of constraining the device within a delivery device, positioning the device proximate a valve; and
the step of deploying the device comprises the step of withdrawing the constraint.

51. A method of decreasing the distance between leaflets of a valve of a subject, said method comprising the step of:
implanting a device proximate a valve of a subject, wherein said device comprises a first arm and a second arm, said first and second arm comprising means for engaging opposing walls of a lumen of a subject.

52. The method according to claim 51 with the added step of compressing the lumen of a subject in order to engage said first and second arm and the opposing walls of a lumen of a subject.

53. The method according to claim 51 wherein said device is implanted in a minimally invasive manner.

54. The method according to claim 51 wherein said device comprises a delivery configuration and a deployed configuration, and said step of implanting the device comprises the steps of percutaneously delivering the device and deploying the device.

55. The method according to claim 54 wherein said step of percutaneously delivering the device comprises the step of constraining the device within a delivery device, positioning the device proximate a valve; and
the step of deploying the device comprises the step of withdrawing the constraint.

56. A method of decreasing the distance between leaflets of a valve of a subject, said method comprising the step of:
implanting a device proximate a valve of a subject, wherein said device comprises means for engaging opposing walls of a lumen of a subject.

57. The method according to claim 56 wherein said device comprises one or more helices.

58. The method according to claim 57 wherein the step of implanting said device comprises rotating one or more of said helices in order to advance one or more of said helices through one or more walls of a lumen of a subject.

59. The method according to claim 51 wherein said method comprises the step of expanding said device in order to engage the opposing walls of a lumen of a subject.

60. A device for implanting an apparatus to decrease the distance between leaflets of a valve, said device comprising a delivery configuration and an expanded configuration, and one or more rails, wherein said one or more rails is substantially linear in said delivery configuration and comprises an apex when in said expanded configuration.

61. A method of decreasing the distance between leaflets of a valve of a subject, said method comprising the steps of:
positioning an apparatus proximate a valve of a subject, wherein said apparatus comprises a first arm and a second arm, a delivery configuration, an expanded configuration and a deployed configuration and means for engaging opposing walls of a lumen of a subject, and said delivery device comprises a delivery configuration and an expanded configuration;
placing said delivery device in its expanded configuration in order to place said apparatus in its expanded configuration and to engage the opposing walls of a lumen of a subject;
returning said delivery device to its delivery configuration and allowing said apparatus to return to its deployed configuration;
retracting said delivery device.

62. The method according to claim 61 wherein said delivery device comprises a delivery configuration and an expanded configuration, and one or more rails, wherein said one or more rails is substantially linear in said delivery configuration and comprises an apex when in said expanded configuration.

63. The apparatus according to claim 39 wherein said apparatus comprises a delivery configuration and a deployed configuration.

64. The apparatus according to claim 63 wherein said deployed configuration comprises a substantially helical configuration.

65. The apparatus according to claim 63 wherein said deployed configuration comprises a first end and a second end, wherein said first end engages the exterior of a wall of a lumen, and said second end engages the exterior of an opposing wall of a lumen of a subject.

66. The apparatus according to claim 65 wherein said first end is generally a mirror image of said second end.

67. The apparatus according to claim 65 wherein said first end is generally a reverse mirror image of said second end.

68. The apparatus according to claim 39 wherein said apparatus a first arm and a second arm and a spring bias disposed therebetween.

69. The apparatus according to claim 65 wherein said first end is oriented in a generally linear relation to said second end.

70. The apparatus according to claim 68 wherein said first arm and said second arm are generally of curvilinear configuration when said apparatus is in its deployed configuration.

71. The apparatus according to claim 32 wherein said means for securing the apparatus comprises one or more sutures.

72. The apparatus according to claim 26 wherein the device comprises shape memory material.

73. The apparatus according to claim 39 wherein the device comprises shape memory material.
